(19) United States
(12) Patent Application Publication
(10) Pub. No.: US 2008/0188756 A1
(43) Pub. Date: Aug. 7, 2008

(54) ULTRASONIC DIAGNOSTIC APPARATUS

(75) Inventor: Takanao FUJIMURA, Sagamihara-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/024,373

(22) Filed: Feb. 1, 2008

(30) Foreign Application Priority Data
Feb. 2, 2007 (JP) 2007-024617

(51) Int. Cl. A61B 8/00

(52) U.S. Cl. 600/459

(57) ABSTRACT

An ultrasonic diagnostic apparatus includes an ultrasonic transducer including an acoustic lens, a piezoelectric element, a backing material, and a housing, a transducer holder that holds the ultrasonic transducer, a transducer side connector fixed in an inside of the ultrasonic transducer, and a holder side connector that is arranged in the transducer holder and connectable to the transducer side connector. Positioning and fixing of the ultrasonic transducer are performed by fitting the ultrasonic transducer in the transducer holder, the holder side connector is movable in a direction perpendicular to a connector connecting direction and is positioned by connection to the transducer side connector to electrically connect the ultrasonic transducer. With this ultrasonic diagnostic apparatus, it is possible and easy to replace the ultrasonic transducer, work for replacing the ultrasonic transducer is easy, and a distal end portion of the ultrasonic diagnostic apparatus incorporated with the ultrasonic transducer can be reduced in size.

### CROSS REFERENCE TO RELATED APPLICATION

This application claims benefit of Japanese Application No. 2007-24617 filed in Japan on Feb. 2, 2007, the contents of which are incorporated by this reference.

### BACKGROUND OF THE INVENTION

1. Field of the Invention

The present invention relates to an ultrasonic diagnostic apparatus such as an ultrasonic endoscope, which is an apparatus for rotating an ultrasonic transducer in a portion to be observed to perform ultrasonic observation.

2. Description of the Related Art

In the past, various ultrasonic diagnostic apparatuses have been proposed concerning an ultrasonic diagnostic apparatus that performs ultrasonic observation by mechanical scanning. For example, an ultrasonic endoscope apparatus disclosed in Japanese Patent Laid-Open No. 2005-130944 includes an ultrasonic diagnostic apparatus (an ultrasonic probe) inserted through an insertion section inserted into a body cavity of an ultrasonic endoscope. The ultrasonic diagnostic apparatus is an apparatus driven to rotate in a state inserted in a body cavity to thereby transmit ultrasound to a portion to be observed in the body cavity and obtain an ultrasound tomographic image.

The ultrasonic diagnostic apparatus in the past includes a distal end cap that also serves as an ultrasonic window at a distal end of an insertion section, an ultrasonic transducer arranged in the distal end cap for outputting an observation ultrasound, and an encoder section and a motor section electrically connected via a slip ring section. The ultrasonic transducer is arranged in the distal end cap section, which is filled with an ultrasound propagation medium (an acoustic medium), in a state in which the ultrasonic transducer can rotate around a support shaft. During ultrasonic observation, the ultrasonic transducer can radiate an ultrasound to the portion to be observed in the body cavity, capture an echo signal of the ultrasound, and obtain an ultrasonic observation image signal.

### SUMMARY OF THE INVENTION

One ultrasonic diagnostic apparatus according to the present invention includes an ultrasonic transducer including an acoustic lens, an electromechanical conversion element, a backing material, and a housing, a transducer holder that holds the ultrasonic transducer, a first connector fixed in an inside of the ultrasonic transducer, and a second connector that is arranged in the transducer holder and connectable to the first connector. Positioning and fixing of the ultrasonic transducer are performed by fitting the ultrasonic transducer in the transducer holder, the second connector is movable in a direction perpendicular to a connecting direction in the transducer holder in a state before connector connection and is positioned by connection to the first connector to electrically connect the ultrasonic transducer.

Another ultrasonic diagnostic apparatus according to the present invention includes an ultrasonic transducer including an acoustic lens, an electromechanical conversion element, a backing material, and a housing, a transducer holder that is driven to rotate in a state of holding the ultrasonic transducer, a cap arranged on an outer side of the ultrasonic transducer, an acoustic medium filled in the cap, a first connector fixed in an inside of the ultrasonic transducer, and a second connector that is arranged in the transducer holder, movable in a direction perpendicular to a connecting direction in the transducer holder in a state before connector connection, and connectable to the first connector. The transducer holder has an external shape corresponding to an external shape of the ultrasonic transducer. The ultrasonic transducer deforms transducer holder elastically and fit in the transducer holder to be positioned and fixed. The second connector is positioned by connection to the first connector to electrically connect the ultrasonic transducer.

Still another ultrasonic diagnostic apparatus according to the present invention includes an ultrasonic transducer including an acoustic lens, an electromechanical conversion element, a backing material, and a housing having a cylindrical external shape portion, a transducer holder that has a cylindrical inner peripheral surface shape portion corresponding to the cylindrical external shape of the housing and holds the ultrasonic transducer, a first connector fixed in an inside of the ultrasonic transducer, and a second connector that is arranged in the transducer holder and supported in a state movable in a direction perpendicular to a connector connecting direction in the transducer holder in a state before connector connection. In the ultrasonic transducer or the transducer holder, a positioning convex portion or concave portion for determining a position in an ultrasound generating direction of the ultrasonic transducer is formed. In the ultrasonic transducer and the transducer holder, the transducer holder side is elastically deformed, the cylindrical external shape portion of the ultrasonic transducer fits in the cylindrical inner peripheral surface shape portion of the transducer holder via the positioning convex portion or concave portion, positioning and fixing in the ultrasound generating direction of the ultrasonic transducer are performed, and the first connector and the second connector are connected to electrically connect the ultrasonic transducer.

Still another ultrasonic diagnostic apparatus according to the present invention includes an ultrasonic transducer including an acoustic lens, an electromechanical conversion element, a backing material, and a housing having a cylindrical external shape portion, a transducer holder that has a cylindrical inner peripheral surface shape portion corresponding to a cylindrical external shape of the housing and holds the ultrasonic transducer, a first connector fixed in an inside of the ultrasonic transducer, and a second connector that is arranged in the transducer holder and movable in a direction perpendicular to a connector connecting direction in the transducer holder in a state before connector connection. In the ultrasonic transducer, a convex step portion for determining a position in an ultrasound generating direction of the ultrasonic transducer is provided. The transducer holder has a fitting portion corresponding to the step portion. In fitting of the ultrasonic transducer and the transducer holder, the transducer holder is elastically deformed and the cylindrical external shape portion of the ultrasonic transducer fits in the cylindrical inner peripheral surface shape portion of the transducer holder in the step portion to be positioned and fixed. The first connector and the second connector are connected to electrically connect the ultrasonic transducer.

Still another ultrasonic diagnostic apparatus according to the present invention includes an ultrasonic transducer including an acoustic lens, an electromechanical conversion element, a backing material, and a housing, a first connector fixed in an inside of the ultrasonic transducer, and a second connector connectable to the first connector. A ground terminal of the first connector is electrically and mechanically fixed to the housing on an outer side of the ultrasonic transducer.

Still another ultrasonic diagnostic apparatus according to the present invention includes an ultrasonic transducer including an acoustic lens, an electromechanical conversion element, a backing material, and a housing, a first connector fixed in the inside of the ultrasonic transducer, and a second connector connectable to the first connector. A wiring portion of the first connector to the ultrasonic transducer is covered with a protection member in order to prevent contact of an electric connection portion with an adjacent terminal.

Still another ultrasonic diagnostic apparatus according to the present invention includes an ultrasonic transducer including an acoustic lens, an electromechanical conversion element, a backing material, and a housing, a first connector fixed in an inside of the ultrasonic transducer, and a second connector connectable to the first connector. A positioning portion for performing positioning of the ultrasonic transducer with respect to the housing is provided on an external shape portion of the first connector.

According to the present invention, it is possible to provide an ultrasonic diagnostic apparatus with high observation accuracy in which an ultrasonic transducer can be replaced alone, work for replacing the ultrasonic transducer is easy, and a distal end portion of the ultrasonic diagnostic apparatus incorporated with the ultrasonic transducer can be reduced in size.

Other characteristics and advantages of the present invention will be made apparent by following explanation.

### BRIEF DESCRIPTION OF THE DRAWINGS

FIG. 1 is a diagram showing a configuration of an ultrasonic endoscope apparatus including an ultrasonic diagnostic apparatus according to an embodiment of the present invention;

FIG. 2 is a perspective view of a distal end portion of an insertion section of an ultrasonic endoscope shown in FIG. 1;

FIG. 3 is a cross-sectional view taken along III-III in FIG. 2 and shows a diagram of a transducer unit housed in the distal end portion viewed from the front;

FIG. 4 is a cross-sectional view taken along IV-IV in FIG. 2 and shows a section along a rotation axis d0 of the transducer unit;

FIG. 5 is a cross-sectional view taken along V-V in FIG. 2 and shows a diagram of the transducer unit viewed from the top;

FIG. 6 is a disassembled perspective view of the transducer unit shown in FIG. 3;

FIG. 7 is a cross-sectional view along a sound axis s0 of a housing and a transducer side connector configuring an ultrasonic transducer shown in FIG. 6;

FIG. 8 is a cross-sectional view taken along VIII-VIII in FIG. 7;

FIG. 9 is a cross-sectional view taken along IX-IX in FIG. 4 and is a cross-sectional view along a connector center axis c0 of the transducer unit;

FIG. 10A is a diagram showing a fitting portion shape of a connector according to a modification for a connector section of the ultrasonic transducer shown in FIG. 6 and is a diagram of a fitting opening portion of a transducer side connector according to the modification viewed from an H direction in FIG. 6; and

FIG. 10B is a diagram of a connector convex portion of a holder side connector according to the modification shown in FIG. 10A viewed from an I direction in FIG. 6.

### DETAILED DESCRIPTION OF THE PREFERRED EMBODIMENTS

Embodiments of the present invention will be hereinafter explained with reference to the accompanying drawings. In the embodiments described below, an ultrasonic diagnostic apparatus is applied to an ultrasonic endoscope.

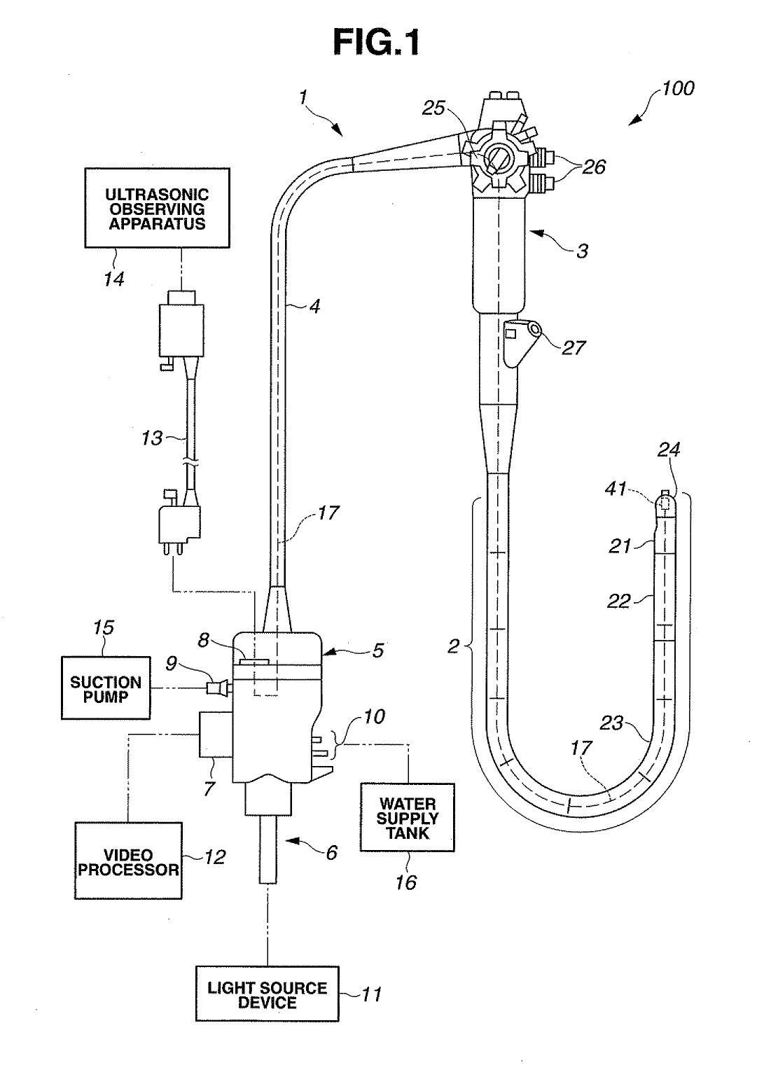

An ultrasonic endoscope apparatus including an ultrasonic diagnostic apparatus according to an embodiment of the present invention is explained with reference to FIGS. 1 to 6.

As shown in FIG. 1, an ultrasonic endoscope apparatus 100 includes, as a main part thereof, an ultrasonic endoscope 1, a light source device 11, a video processor 12, an ultrasonic observing apparatus 14, a suction pump 15, and a water supply tank 16.

The ultrasonic endoscope 1 includes, as a main part thereof, an elongated insertion section 2 inserted into a body cavity, an operation section 3 also serving as a gripping section provided in a proximal end portion in an inserting direction of the insertion section 2, a universal cord 4 having flexibility extending from, for example, a proximal end portion side in the inserting direction of the operation section 3, and a scope connector 5 in which an extending end of the universal cord 4 is provided.

In the scope connector 5, a light source connector 6, an electrical connector 7, an ultrasonic connector 8, a suction mouthpiece 9, and air-supply and water supply mouthpieces 10 are provided.

The light source device 11 that supplies illumination light is detachably attachable to the light source connector 6. The video processor 12 that performs various kinds of signal processing and the like is detachably attachable to the electrical connector 7 through a not-shown signal cable.

The ultrasonic observing apparatus 14 is detachably attachable to the ultrasonic connector 8 through an ultrasonic cable 13. The suction pump 15 is detachably attachable to the suction mouthpiece 9 through a not-shown suction tube. The water supply tank 16 is detachably attachable to the air-supply and water-supply mouthpieces 10 through not-shown air-supply and water-supply tubes.

The ultrasonic observing apparatus 14 performs various kinds of operation control for the ultrasonic endoscope 1. For example, the ultrasonic observing apparatus 14 performs driving control for an ultrasonic transducer 41 described later and an operation for performing signal processing of an electric signal acquired by the driving control for the ultrasonic transducer 41 to generate a video signal.

The video signal generated by the ultrasonic observing apparatus 14 is outputted to a not-shown display device that configures the ultrasonic endoscope apparatus 100. As a result, an ultrasonic image is displayed on a screen of the display device that receives the video signal.

In the insertion section 2 of the ultrasonic endoscope 1, in order from a distal end side thereof, a distal end rigid portion 21 formed of a rigid member, a bending portion 22 formed to be bendable in, for example, an up/down direction and a left/right direction, and a flexible tube portion 23 that is long and has flexibility are connected in series.

In the operation section 3, a bending operation knob 25 for performing bending operation of the bending portion 22 and air-supply and water-supply buttons 26 for performing air-supply and water-supply operation and suction operation are provided.

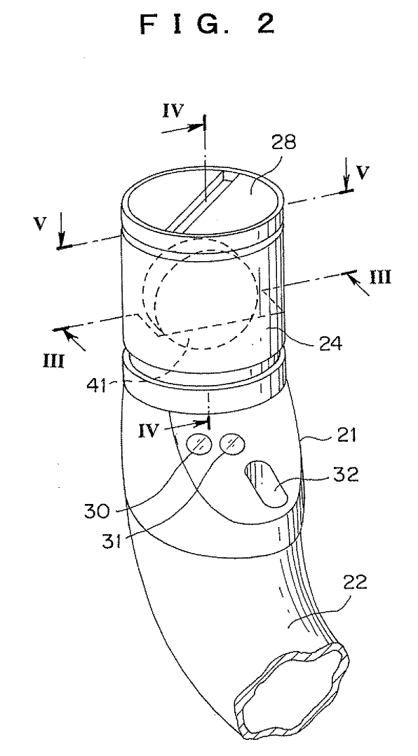

As shown in FIG. 2, an illumination lens 30, an observation lens 31, and a treatment instrument opening 32 are provided in setting positions on an outer peripheral side surface of the distal end rigid portion 21.

The illumination lens 30 emits illumination light, which is supplied from the light source device 11, to an observation region as a region to be inspected in a body cavity through a not-shown light guide inserted through the universal cord 4, the operation section 3, and the insertion section 2. The observation region in the body cavity is illuminated by the illumination light emitted from the illumination lens 30.

The observation lens 31 configures an observation unit together with not shown plural object lenses provided in the distal end rigid portion 21 and a not-shown image-pickup device such as a charge coupled device provided in a image-forming position of the object lens.

An optical image of the observation region observed by the observation lens 31 and the object lens is, after being image-formed on an image-pickup surface of the image-pickup device, photoelectrically converted into an electric signal by the image-pickup device. Thereafter, the electric signal is transmitted to the video processor 12 through a not-shown image-pickup cable extending from the image-pickup device and inserted through the insertion section 2, the operation section 3, and the universal cord 4 and the electrical connector 7.

The video processor 12 applies signal processing to the transmitted electric signal to thereby generate a standard video signal and outputs the video signal to the not-shown display device. As a result, an endoscope observation image is displayed on a screen of the display device.

The treatment instrument opening 32 is an opening on the distal end rigid portion 21 side of a treatment instrument insertion line 33 provided in the insertion section 2 and the operation section 3 and is an opening for projecting the treatment instrument, which is inserted into the treatment instrument insertion line 33 from a treatment instrument entrance 27, to the region to be inspected in the body cavity.

Further on an inserting direction distal end side than the illumination lens 30, the observation lens 31, and the treatment instrument opening 32 of the distal end rigid portion 21, as shown in FIG. 2, a cylindrical cap 24 that also serves as an ultrasonic observation window and is formed of, for example, polyethylene is mounted. In the cap 24, the ultrasonic transducer 41 for observation ultrasound oscillation and echo signal reception of an ultrasonic transducer unit 40 is arranged in a filled ultrasound propagation acoustic medium 75 in a rotatable state.

The insertion section 2 of the ultrasonic endoscope 1 is used to pass a pharynx portion of a subject. Therefore, it is preferable that a diameter of the cap 24 is as small as possible, for example, equal to or smaller than 20 mm.

When the insertion section 2 is inserted into the body cavity, the cap 24 comes into contact with a body cavity wall in the body cavity via a cap outer side medium such as water and body fluid through which ultrasound passes at acoustic velocity of about 1500 m/sec.

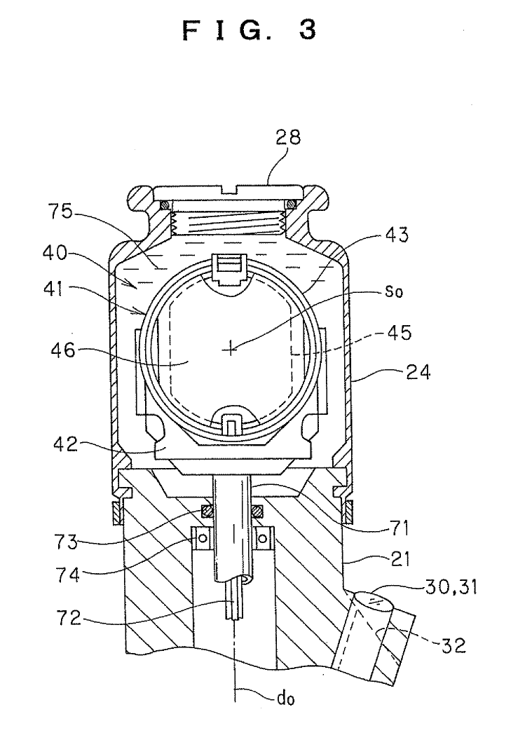

A detailed configuration of the ultrasonic transducer unit 40 is explained with reference to FIGS. 3 to 6 and FIGS. 7 to 9.

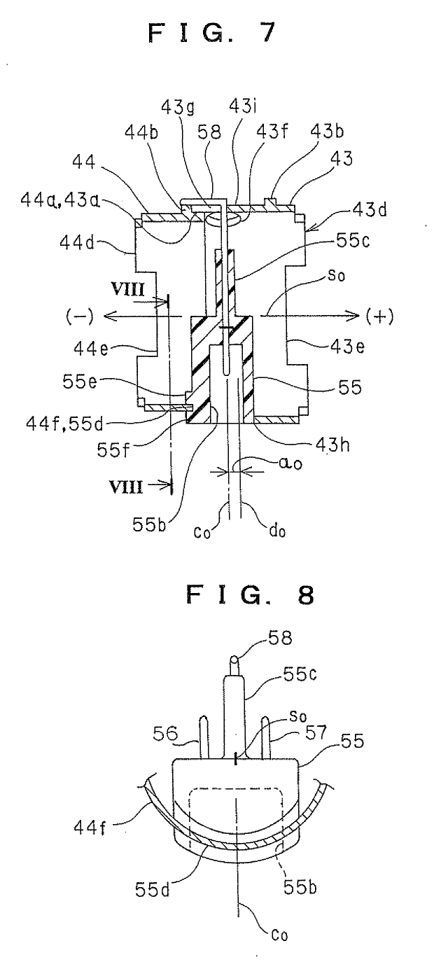

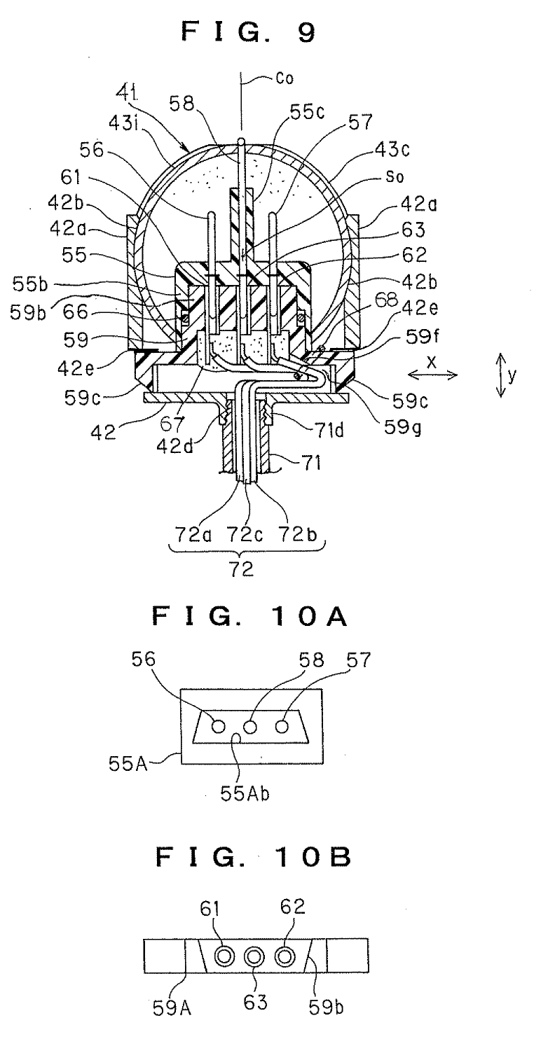

FIG. 7 is a cross-sectional view along a sound axis s0 of a housing and a transducer side connector of an ultrasonic transducer configuring the ultrasonic transducer unit. FIG. 8 is a cross-sectional view taken along VIII-VIII in FIG. 7. FIG. 9 is a cross-sectional view along a connector center axis c0 of the ultrasonic transducer.

The sound axis s0 is a radial sound axis of ultrasounds of low and high frequencies from acoustic lenses 46 and 49 described later. A sound axis direction on a side on which lower-frequency ultrasound is radiated (the acoustic lens 46 side) is set as a +s0 direction. The direction is a front side of the ultrasonic transducer. A sound axis direction on a side on which higher-frequency ultrasound is radiated (the acoustic lens 49 side) is set as a −s0 direction. The direction is a rear side of the ultrasonic transducer.

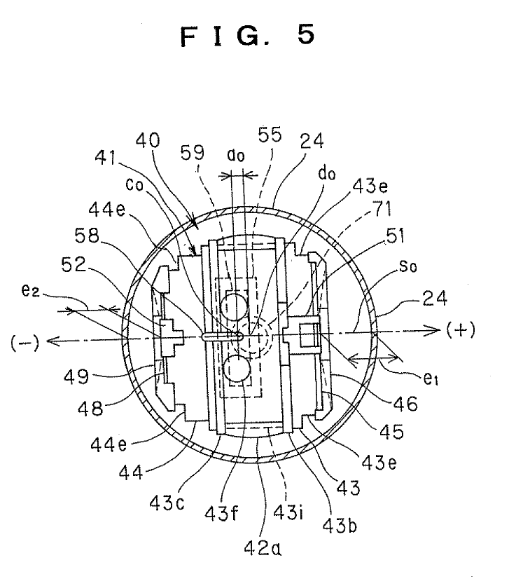

The ultrasonic transducer is driven to rotate by a transducer holder. A rotation axis of the ultrasonic transducer is d0. The rotation axis d0 passes through and is orthogonal to the sound axis s0 as described later. The connector center axis c0 indicates a center axis of an electrical connection connector of the ultrasonic transducer. As described later, the connector center axis c0 is parallel to the rotation axis d0 but deviates by an eccentricity amount a0 (FIGS. 5 and 7).

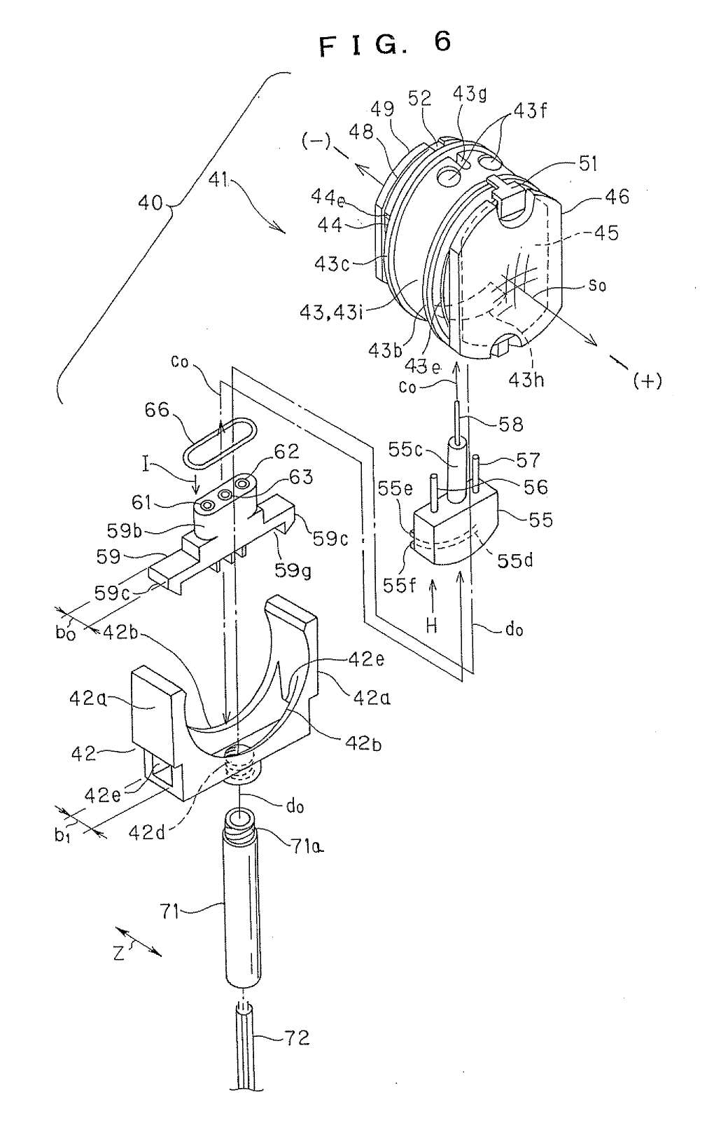

The ultrasonic transducer unit 40 includes, as shown in FIG. 6, the ultrasonic transducer 41, a transducer side connector 55 as a first connector for electrical connection of the transducer and a holder side connector 59 as a second connector, an O ring 66 as a packing material inserted between the connectors 55 and 59, a transducer holder 42 that detachably holds the ultrasonic transducer 41, a driving shaft 71 that is locked to the transducer holder 42 and serves as a rotation axis of the ultrasonic transducer 41, a ball bearing 74 and a water-proof O ring 73 for supporting the driving shaft 71 to be capable of driving with respect to the distal end rigid portion 21, and a transducer lead wire 72 electrically connected to the ultrasonic transducer 41 through the connector.

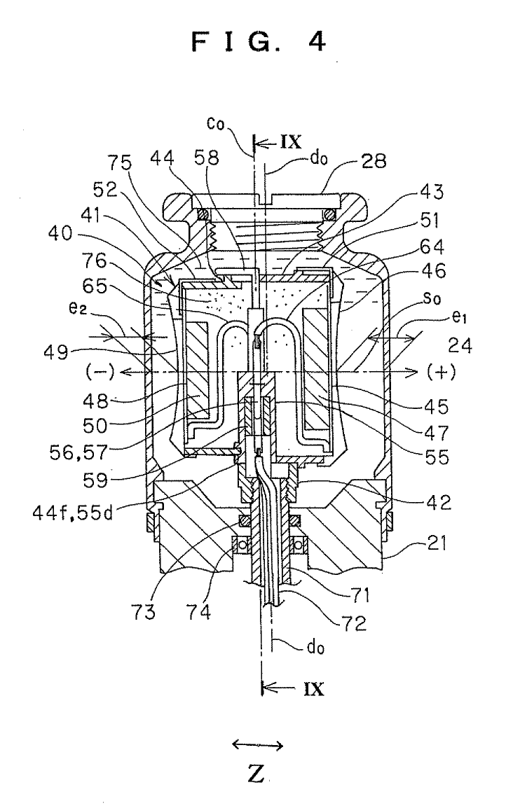

The ultrasonic transducer 41 has a substantially columnar external shape and mainly includes a piezoelectric element 45 as an electromechanical conversion element for high-frequency ultrasound excitation, an acoustic lens 46 of a concave shape on the surface for radiating ultrasound excited by the piezoelectric element 45, a backing material 47 for absorbing ultrasonic vibration to a rear side of the piezoelectric element 45, a piezoelectric element 48 as an electromechanical conversion element for low-frequency ultrasound excitation, an acoustic lens 49 of a concave shape on the surface for radiating ultrasound excited by the piezoelectric element 48, a backing material 50 for absorbing ultrasonic vibration to a rear side of the piezoelectric element 48, and housings 43 and 44 in which the piezoelectric elements, the acoustic lenses, the backing materials, and the transducer side connector 55 are incorporated (FIG. 4). Both the piezoelectric elements 45 and 48 have a substantially elliptical shape and dimensions in an up/down direction (a distal end direction) thereof are larger than dimensions in a width direction (a rotating direction) thereof. An external shape of the piezoelectric element 45 is larger than that of the piezoelectric element 48. Gilding for electrodes is applied to the front and rear surfaces of the piezoelectric elements 45 and 48. Front surface sides (outer sides) are common electrodes and rear surface sides (inner sides) are plus electrodes. The piezoelectric elements 45 and 48 are locked to a front end face 43d and a rear end face 44d of the housings 43 and 44.

The piezoelectric element 48 excites higher-frequency ultrasound and the piezoelectric element 45 excites lower-frequency ultrasound. The ultrasounds excited by the piezoelectric elements are radiated to respective observation regions via the acoustic lenses 46 and 49 described later. The piezoelectric elements 45 and 48 receive echo signals for observation image creation from the observation regions.

The acoustic lenses 46 and 49 are acoustic lenses of a concave shape on the surface that are made of a plastic material such as acrylic resin and have external shapes corresponding to the external shapes of the piezoelectric elements 45 and 48, respectively. The acoustic lenses 46 and 49 are formed on outer sides of the housings 43 and 44 and front and rear surfaces of the piezoelectric elements 45 and 48 by integral molding. The acoustic lenses 46 and 49 radiate the ultrasounds excited by the piezoelectric elements 45 and 48 to observation regions in different focus positions, respectively.

The housings 43 and 44 are metal members of a cylindrical shape, both of which have the sound axis s0 as axes (in an incorporated state). The housings 43 and 44 are integrally coupled by fitting an outer peripheral surface 44a in a cylindrical inner peripheral surface 43a (FIG. 7).

In one housing 43, a notch 43e arranged on the front surface side, positioning convex portions 43b and 43c that project along an outer peripheral direction and form a circumferential groove of predetermined width, two resin injection ports 43f arranged in upper parts on the distal end side between the convex portions 43b and 43c, a notch 43g for connector pin insertion arranged between the resin injection ports 43f and in a position of the connector center axis c0, and an opening portion 43h of a notch shape for connector attachment in a lower portion (the proximal end side) are provided.

A center axis that is in the center of the width of the positioning convex portions 43b and 43c and orthogonal to the sound axis s0 in the up/down direction coincides with an axis of a driving shaft 71 described later in an assembled state of the ultrasonic transducer unit 40 and becomes the rotation axis do. The notch 43e is a notch having a dimension corresponding to the width of the piezoelectric element 45.

In the other housing 44, a bumping convex portion 44b along an outer peripheral direction of the housing 44 and a notch 44e arranged on a rear surface side thereof are provided. The notch 44e is a notch having a dimension corresponding to the width of the piezoelectric element 48.

The backing materials 47 and 50 are foam resin members for absorbing ultrasonic vibration from inner surface sides of the piezoelectric elements 45 and 48. Both the backing materials 47 and 50 are arranged in close contact with respective inner sides of the piezoelectric elements 45 and 48 in the housings 43 and 44.

The transducer side connector 55 is a resin member. As shown in FIGS. 6 and 7, three connector pins projecting along the connector center axis c0 or vertically projecting in parallel are locked to the transducer side connector 55. A connector fitting opening 55b in which the holder side connector 59 can be fit is provided on a lower side (a proximal end side) of the transducer side connector 55. An arcuate groove 55d as a connector positioning portion formed between convex portions 55e and 55f is provided on a surface on the housing 44 side of the transducer side connector 55.

The three connector pins include a common electrode connector pin 58 arranged in the center portion and plus electrode connector pins 56 and 57 for the piezoelectric elements 45 and 48 arranged on both sides. The connector pin 58 is covered with an insulating protection portion 55c formed of an insulating member up to predetermined height of the pin. The insulating protection portion 55c is provided in order to prevent the common electrode connector pin 58 from coming into contact with connection lead wires soldered to the plus electrode connector pins 56 and 57 on both the sides.

When the ultrasonic transducer 41 having the configuration described above is assembled, first, the backing materials 47 and 50 are arranged in a state of contact with the inner sides of the piezoelectric elements 45 and 48, respectively, in the housings 43 and 44 in which the acoustic lenses 46 and 49 are integrally molded on the front and rear surfaces thereof in a state in which the piezoelectric elements 45 and 48 are attached to the front and rear end face portions 43d and 44d, respectively.

Therefore, in the housings 43 and 44, the outer peripheral surface 44a and the inner peripheral surface 43a are fit and the transducer side connector 55 is inserted into the opening portion 43h for connector attachment on the lower side of the housing 43. At that point, a circumferential end 44f of the housing 44 is engaged in the arcuate groove 55d of the transducer side connector 55 and positioned.

However, prior to the fitting of the housings 43 and 44, as shown in FIG. 4, the plus electrode on the inner side of the piezoelectric element 45 and the connector pin 56 are connected and the plus electrode on the inner side of the piezoelectric element 48 and the connector pin 57 are connected by soldering via lead wires 64 and 65, respectively. In a state in which the connector 55 is inserted, the common electrode connector pin 58 is inserted through the notch 43g of the housing 43 and projected upward.

Resin 76 is filled in the inside of the housings 43 and 44 from the resin injection ports 43f in a fitted state of the housings 43 and 44. Portions other than the resin injection ports 43f are sealed in the fitted state of the housings 43 and 44. After the resin is solidified, the common electrode connector pin 58 projected upward is bent to the housing 44 side and soldered on the bumping convex portion 44b, whereby electrical connection and mechanical fixing are performed. A connection piece 51 connected to the common electrode on the outer surface of the piezoelectric element 45 is soldered to the outer periphery of the positioning convex portion 43b of the housing 43. A connection piece 52 connected to the common electrode on the outer surface of the piezoelectric element 48 is soldered to the outer periphery of the housing 44. The ultrasonic transducer 41 is completed in this soldered state.

In the ultrasonic transducer 41, the connector center axis c0 in the center of the transducer side connector 55 (the position of the common electrode connector pin 58) passes a position in the middle between both the end faces 43d and 44d (surfaces on which the piezoelectric elements 45 and 48 are attached) of the housings 43 and 44 and passes the sound axis s0.

In molding of the acoustic lenses 46 and 49, the convex portion 43b of the housing 43 and the convex portion 44b of the housing 44 also play a function of preventing the resin from invading a cylindrical outer peripheral portion 43i as a housing fitting portion. The convex portions 43b and 44b also function as protectors for preventing the acoustic lenses 46 and 49 from being scratched. Moreover, when the resin 76 is injected into the housing 43 from the resin injecting ports 43f, the convex portions 43b and 43c of the housing 43 control outflow of the resin 76 to the outside and prevent the resin 76 from adhering to the acoustic lenses 46 and 49.

The transducer holder 42 is a metal member of a substantially C shape that detachably holds the ultrasonic transducer 41. As shown in FIG. 6 and the like, the transducer holder 42 includes an inner peripheral portion 42b for ultrasonic transducer holding that is a portion fit in the cylindrical outer peripheral portion 43i of the housing 43 and an upper part of which is notched, transducer gripping portions 42a opposed on both outer sides of the inner peripheral portion 42b, side opening portions 42e pierced through in both width directions below the gripping portions 42a, and a driving shaft screwing portion 42d arranged in a lower side portion.

The width in the sound axis s0 direction of the transducer gripping portion 42a has a dimension that allows the transducer gripping portion 42a to be engaged without a gap between the positioning convex portions 43b and 43c of the housing 43.

The inner peripheral portion 42b of the transducer holder 42 is formed in an arc substantially around the sound axis s0 slightly smaller than the cylindrical outer peripheral portion 43i of the housing 43. An arcuate angle thereof is always equal to or larger than 180°.

The ultrasonic transducer 41 can be mounted on the transducer holder 42 in a mechanical press-fit state by inserting, while elastically deforming outward, the transducer gripping portion 42a of the transducer holder 42 between the positioning convex portions 43b and 43c of the housing 43 on the ultrasonic transducer 41 side and fitting the inner peripheral portion 42b of the transducer holder 42 in the outer peripheral portion 43i of the housing 43.

In the present embodiment, the positioning convex portions 43b and 43c are arranged on the housing 43 side. However, instead of this, it is also possible to form the cylindrical outer peripheral portion 43i of the transducer holder 42 with a belt-like convex surface portion, form a fitting concave portion that fits in the convex surface portion on the transducer holder 42 side, and press-fit and hold the ultrasonic transducer 41 in the transducer holder 42 in the same manner.

Both-side extending portions 59c described later are slidably fit in the side opening portion 42e of the transducer holder 42 in a state in which the holder side connector 59 is assembled.

The holder side connector 59 is a connector member of a T shape as shown in FIGS. 6 and 9. The holder side connector 59 includes a connector fitting convex portion 59b that can be fit without a gap in the connector fitting opening 55b of the transducer side connector 55, the both-side extending portions 59c, a wiring space 59g for wire holding of a concave shape in which a connection lead wire 72 can be arranged in a loosened state on an inner side of the extending portions 59c, and an inclined groove portion 59f as a holding portion for the connection lead wire 72.

Three receptacles are inserted and locked in the connector fitting convex portion 59b. A common electrode receptacle 63 is locked along the sound axis s0 in the center and plus electrode receptacles 61 and 62 are locked along a direction parallel to the sound axis s0 on both sides of the common electrode receptacle 63. Lead wire soldering tabs projecting to a lower part of the connector fitting convex portion 59b are provided in the receptacles 61, 62, and 63, respectively.

The holder side connector 59 is attached to the transducer holder 42 by slidably inserting the both-side extending portions 59c in the side opening portions 42e. Therefore, the holder side connector 59 is supported to be movable by a small amount in a left/right direction (an x direction in FIG. 9) orthogonal to a connector connecting direction coinciding with the connector center axis c0 (in other words, a state in which the holder side connector 59 is movable according to a connector connecting position). However, movement of the holder side connector 59 in an up/down direction (a y direction in FIG. 9) along the connector center axis c0 is regulated (in other words, movement in a connecting inserting and removing direction is regulated).

The driving shaft 71 is a rigid tubular member. A screw portion 71 a is provided at an upper end thereof. The screw portion 71 a is screwed in the screw portion 42d of the transducer holder 42. The driving shaft 71 is integrated with the transducer holder 42. In the integrated state, the rotation axis d0 as the axis of the driving shaft 71 deviates to a front side (a plus direction of the sound axis so) by an eccentricity amount a0 with respect to the connector center axis c0 of the transducer holder 42.

The transducer lead wire 72 is inserted through the inside of the tube of the driving shaft 71 screwed in the transducer holder 42. The lead wire 72 includes plus electrode lead wires 72a and 72b and a common electrode lead wire 72c, which are connected to the tabs of the receptacles 61, 62, and 63 of the holder side connector 59 by soldering, respectively.

As shown in FIG. 9, after the lead wire 72 is soldered, resin 67 is filled around tab soldered portions of the receptacles 61, 62, and 63 and solidified to protect the tab soldered portions. The lead wire 72 is kept folded and housed in the wiring space 59g below the holder side connector 59 or the inclined groove 59f, tied to one of the both-side extending portions 59c by a bundling band 68 as a fastening member, or a sting, a rubber band, or the like, and integrally solidified.

As described above, the lead wire 72 is connected by soldering. The connector 59 with the tab portions of the receptacles 61, 62, and 63 solidified by resin is mounted to the transducer holder 42 by inserting the both-side extending portions 59c in the side opening portions 42e of the transducer holder 42. In the transducer holder 42, the driving shaft 71 is screwed in the screw portion 42d from the down side and integrated.

The ultrasonic transducer 41 is mounted in a detachable state by pressing and fitting the outer peripheral portion 43i of the housing 43 in the inner peripheral portion 42b of the transducer holder 42 assembled with the driving shaft 71 and the holder side connector 59 as described above.

When the ultrasonic transducer 41 is mounted on the transducer holder 42, at the same time, the connector fitting convex portion 59b of the holder side connector 59 is fit in the connector fitting opening 55b of the transducer side connector 55 to electrically connect the connector pins and the receptacles. When the connector 59 is slightly moved by fitting the connector fitting convex portion 59b as described above, it is possible to prevent a stress from acting on the soldered portions of the lead wire and the receptacles.

The width b0 of the connector 59 is set smaller than the width b1 of the transducer holder 42 (FIG. 6). Consequently, the connector 59 is slightly movable in a Z direction in FIG. 6 as well.

In the connector fitting (connection) state described above, since the holder side connector 59 is movable in the x z direction as described above, even if the holder side connector 59 is misaligned, the holder side connector 59 can be fit in the transducer side connector 55 in a state in which the centers thereof are aligned. When the holder side connector 59 moves in the y direction as a connector pressing direction, the both-side extending portions 59c of the connector 59 are fit in the side opening portions 42e of the transducer holder 42. Therefore, the both-side extending portion 59c acts as a stopper portion in a connector fitting direction. It is possible to surely prevent the connector 59 from coming off.

When the connectors 55 and 59 are fit, the O ring 66 for sealing is inserted in the connector fitting portion as a packing material (FIG. 6). A proximal end side of the lead wire 72 is electrically connected to the signal cable 17 in the universal cord 4 through the insertion section 2.

When the ultrasonic transducer 41 and the transducer holder 42 are assembled, the holder side connector 59 may dimensionally stop up in the space of the transducer holder 42 in the axial direction. In such a case, since arm portions of both-side extending portions 59c are elastically deformed vertically, the stop-up can be allowed and the holder side connector 59 can be naturally housed in the transducer holder 42.

When the connectors 55 and 59 are disconnected, since movement of the holder side connector 59 in the y direction is regulated as described above, it is possible to easily remove the connector 59.

The ultrasonic transducer unit 40 with the ultrasonic transducer 41 mounted on the transducer holder 42 is attached to the distal end portion of the distal end rigid portion 21. More specifically, as shown in FIGS. 3 and 4, the O ring 73 for sealing is inserted at the distal end portion of the distal end rigid portion 21 and the driving shaft 71 on the transducer holder 42 side is attached to the distal end portion via the ball bearing 74 in a rotatable state.

The ultrasonic transducer 41 is locked to the distal end portion of the distal end rigid portion 21 while being covered with the cap 24 as described above. The acoustic medium 75 is filled in the inside of the cap 24 and the distal end of the cap 24 is closed by a plug 28.

The acoustic medium 75 improves rotation lubricity of the ultrasonic transducer 41. Generation of air bubbles in the acoustic medium 75 during rotation of the ultrasonic transducer 41 is prevented. For that purpose, an oily medium such as glycerin, in which acoustic velocity of ultrasound is lower than that in the cap outer side medium, is applied.

In the attached state of the ultrasonic transducer 40, the rotation axis do as the axis of the driving shaft 71 coincides with the cylindrical center axis of the cap 24 (in other words, orthogonal to the sound axis s0).

Separation distances between the respective front surfaces of the acoustic lenses 46 and 49 and the inner peripheral surface of the cap 24 need to be reduced to limits, respectively, in order to suppress attenuation of ultrasonic outputs. On the other hand, as described above, the widths in the rotating direction of the piezoelectric element 45 and the acoustic lens 46 on the low-frequency side are set wider than the widths in the rotating direction of the piezoelectric element 48 and the acoustic lens 49 on the high-frequency side. Therefore, a separation distance e1 between the front surface of the acoustic lens 46 and the inner peripheral surface of the cap 24 is larger than a separation distance e2 between the front surface of the acoustic lens 49 and the inner peripheral surface of the cap 24.

Therefore, in order to satisfy the positional relation described above, as described above, it is necessary to inevitably make the connector center axis c0 as the center axis of the ultrasonic transducer 41 eccentric with respect to the rotation axis d0. An eccentricity amount a0 thereof is about [e1−e2]×½.

In the ultrasonic transducer unit 40, when it is necessary to replace the ultrasonic transducer 41, first, the cap 24 is removed from the distal end rigid portion 21. When the ultrasonic transducer 41 is pulled out from the transducer holder 42 while the transducer gripping portion 42a is widened, the transducer side connector 55 and the holder side connector 59 are disconnected. At the same time, the ultrasonic transducer 41 is completely separated from the transducer holder 42. At that point, the holder side connector 59 is held by the both side opening-portions 42e of the transducer holder 42. Therefore, the connectors 55 and 59 can be surely separated.

The ultrasonic transducer unit 40 can be reproduced by fitting the new ultrasonic transducer 41 in the transducer holder 42.

When ultrasonic observation of the region to be inspected is performed by the ultrasonic endoscope apparatus 100 having the configuration explained above, the ultrasonic transducer 41 radiates, while rotating around the rotation axis d0, higher-frequency or lower-frequency ultrasound on the region to be inspected. After the radiation, the ultrasonic transducer 41 receives ultrasound reflected from the region to be inspected and converts the ultrasound into an electric signal. The electric signal is transmitted to the ultrasonic observing apparatus 14 through the signal cable 17, the ultrasonic connector 8, and the ultrasonic cable 13.

The ultrasonic observing apparatus 14 generates an ultrasonic image signal by applying signal processing to the transmitted electric signal and outputs the ultrasonic image signal to the not-shown display device. As a result, an ultrasonic image is displayed on the screen of the display device.

The ultrasonic transducer 41 includes the piezoelectric elements 45 and 48 having different frequencies. When the ultrasonic transducer 41 rotates, the piezoelectric elements 45 and 48 are opposed to the region to be inspected and the transducer radiates ultrasound to the region to be inspected from the piezoelectric element 45 or 48. Therefore, it is possible to obtain satisfactory ultrasonic images of regions to be inspected in positions at different distances from the ultrasonic transducer 41.

As explained above, with the ultrasonic transducer unit 40 that configures the ultrasonic diagnostic apparatus according to the present embodiment, it is possible to provide an ultrasonic diagnostic apparatus with high observation accuracy in which the ultrasonic transducer 41 can be replaced alone, work for replacing the ultrasonic transducer is easy, and a distal end portion of an ultrasonic probe incorporated with the ultrasonic transducer can be reduced in size.

More specifically, with the ultrasonic transducer unit 40 according to the present embodiment, the ultrasonic transducer 41 is fit in the transducer holder 42 to be positioned in a sound axis direction and mounted. When the ultrasonic transducer 41 is fit in the transducer holder 42, the transducer side connector 55 and the holder side connector 59 are connected to electrically connect the ultrasonic transducer 41 and the transducer holder 42. Therefore, it is possible to simultaneously and surely perform the positioning of the ultrasonic transducer 41 and the connection of the connectors 55 and 59. The holder side connector 59 is movably supported in a state before the connector connection and fixedly supported after the connector connection. Therefore, the electrical connection is performed reliably in a natural state.

Moreover, when the ultrasonic transducer 41 is fit in the transducer holder 42 and positioned, since the transducer holder 42 is elastically deformed to fit the ultrasonic transducer 41 therein. Therefore, it is possible to position the ultrasonic transducer 41 without backlash and accurately. In a state in which the ultrasonic transducer 41 and the transducer holder 42 are fit and held, the rotation axis of the ultrasonic transducer 41 and the axis of the connectors 55 and 59 are eccentric. In other words, the rotation axis of the ultrasonic transducer 41 is eccentric with respect to the center of the transducer holder 42. Therefore, the both-side extending portions 59c that fit in the transducer holder 42 are provided in the holder side connector 59 for which a suitable arrangement can be adopted with respect to the ultrasonic transducer 41 having an asymmetrical shape. Therefore, when the ultrasonic transducer 41 is pulled out from the transducer holder 42, the both-side extending portions 59c function as stopper portions and the holder side connector 59 does not come off the transducer holder 42.

The both-side extending portion 59c provided in the holder side connector 59 has a shape elastically deformable with respect to a connector fitting direction (a pressing direction). Therefore, generation of an abnormal pressing force due to dimensional stop-up during connector connection is relaxed.

The O ring 66 for sealing is fit in the fitting portion of the transducer side connector 55 and the holder side connector 59. Therefore, the acoustic medium 75 is prevented from intruding into the inside of the ultrasonic transducer 41.

The wiring space 59g for wire holding of a concave shape is provided on the lower inner side of the both-side extending portion 59c of the holder side connector 59. Therefore, the connection lead wire 72 can be arranged even in a loosened state, wiring work is easy, the wiring can be performed in a state in which an abnormal force does not act on the lead wire.

The lead wire 72 connected to the receptacles as the connection terminal portions of the holder side connector 59 is fixed to and integrated with the extending portions 59c by a bundling band in a state held in the wiring space 59g. Therefore, even if the holder side connector 59 slightly moves, it is possible to prevent a force from acting on the solder connecting portion of the lead wire 72.

The inclined groove portion 59f as the holding portion for the connection lead wire 72 is provided at a corner of the wiring space 59g for wire holding of a concave shape on the lower inner side of the both-side extending portion 59c of the holder side connector 59. Therefore, it is possible to naturally hold the connection lead wire 72.

The cylindrical external shape portion of the housing of the ultrasonic transducer 41 and the cylindrical inner peripheral surface shape portion of the transducer holder 42 are fit and positioning in the sound axis direction with respect to the transducer holder 42 is performed by the positioning convex portions 43b and 43c of the ultrasonic transducer 41. Therefore, the ultrasonic transducer 41 can be accurately positioned in an ultrasound generating direction. The positioning convex portions 43b and 43c may be provided in the transducer holder 42. Alternatively, the positioning may be performed by a concave portion provided in the ultrasonic transducer 41 or the transducer holder 42. In both the cases, connection of the connectors is simultaneously performed during the fitting described above. When the positioning convex portions 43b and 43c are formed of step portions of a convex shape, effects same as those described above can be realized.

The common electrode connector pin 58, which is a ground terminal of a transducer side connector for electrical connection of the ultrasonic transducer 41 and the transducer holder 42, is electrically and mechanically fixed in the outer peripheral portion of the housing of the ultrasonic transducer 41. Therefore, it is possible to simultaneously perform the fixing of the connectors and the electrical connection of the connector pins.

The insulating protection portion 55c is provided in a root portion of the common electrode connector pin 58 of the transducer connector 55. Therefore, the common electrode connector pin 58 is prevented from coming into contact with the lead wire connected to the plus electrode connector pins 56 and 57 arranged on both the outer sides of the common electrode connector pin 58.

In the transducer side connector 55, the positioning arcuate groove 55d that engages with the housing circumferential end 44f when the ultrasonic transducer 41 is incorporated in the housing is provided. Therefore, it is possible to accurately position the transducer side connector 55 with respect to the housing.

A connector portion having a reverse insertion preventing function is explained as a modification of the connector portion of the ultrasonic transducer 41 according to the embodiment described above.

FIGS. 10A and 10B are diagrams showing a fitting portion shape of the connector according to the present modification. FIG. 10A is a diagram of a fitting opening portion 55Ab of a transducer side connector 55A according to the present modification viewed from an H direction in FIG. 6. FIG. 10B is a diagram of a connector convex portion 59Ab of a holder side connector 59A according to the present embodiment viewed from an I direction in FIG. 6.

The fitting opening portion 55Ab of the transducer side connector 55A has a vertically symmetrical trapezoidal shape as shown in FIG. 10A. The connector convex portion 59Ab of the holder side connector 59A also has a vertically symmetrical trapezoidal shape and can be fit in the fitting opening portion 55Ab only in a specific fitting posture. Therefore, the connectors 55A and 59A cannot be reversely inserted and normal connection is always performed. The same effect can be obtained even if the fitting portion shape of the connectors is a horizontally symmetrical shape.

In the structure applied to the ultrasonic transducer 41 according to the present embodiment, the ultrasonic transducer 41 is mechanically pressed and held in the transducer holder 42 and detachably attached thereto. As a modification of the attachment structure of the ultrasonic transducer 41 detachably attachable to the transducer holder 42, the structure in which a mechanical lock mechanism is also used for connection of a transducer side connector and a holder side connector can be proposed.

In modification described above, a lock pawl is provide in addition to fitting of a fitting portion and a convex portion of the transducer side connector and the holder side connector to realize a connection state of both the connectors and mechanical holding of the ultrasonic transducer. In the present modification, it is unnecessary to provide a transducer gripping portion in a transducer holder.

The ultrasonic diagnostic apparatus according to the present invention can be used as an ultrasonic diagnostic apparatus with high observation accuracy in which an ultrasonic transducer can be replaced alone, work for replacing the ultrasonic transducer is easy, and a distal end portion of the ultrasonic diagnostic apparatus incorporated with the ultrasonic transducer can be reduced in size.

The present invention is not limited to the embodiments described above. At an implementation stage, various modifications can be carried out without departing from the spirit of the invention. Moreover inventions at various stages are included in the embodiments. Various inventions can be extracted according to appropriate combinations in the disclosed plural requirements.

What is claimed is:

1. An ultrasonic diagnostic apparatus comprising:
an ultrasonic transducer including an acoustic lens, an electromechanical conversion element, a backing material, and a housing;
a transducer holder that holds the ultrasonic transducer;
a first connector fixed in an inside of the ultrasonic transducer; and
a second connector that is arranged in the transducer holder and connectable to the first connector, wherein
positioning and fixing of the ultrasonic transducer are performed by fitting the ultrasonic transducer in the transducer holder, the second connector is movable in a direction perpendicular to a connecting direction in the transducer holder in a state before connector connection and is positioned by connection to the first connector to electrically connect the ultrasonic transducer.

2. The ultrasonic diagnostic apparatus according to claim 1, wherein a rotation axis of the ultrasonic transducer and a center axis of the first connector and the second connector are eccentric.

3. The ultrasonic diagnostic apparatus according to claim 1, wherein the second connector has, between the second connector and the transducer holder, a stopper portion for preventing the second connector from coming off the transducer holder when the first connector and the second connector are disconnected.

4. The ultrasonic diagnostic apparatus according to claim 1, wherein an elastic deforming portion is provided in the second connector to make it possible to relax a pressing force of the first connector applied to the second connector in a fitting state of the ultrasonic transducer and the transducer holder.

5. The ultrasonic diagnostic apparatus according to claim 1, wherein a packing material is arranged in a fitting portion of the first connector and the second connector.

6. The ultrasonic diagnostic apparatus according to claim 1, wherein a wire holding space portion for holding wiring is provided in the second connector.

7. The ultrasonic diagnostic apparatus according to claim 6, wherein the wiring connected to a connection terminal portion of the second connector is fixed to the second connector by a fastening member in a state held in the wire holding space portion.

8. The ultrasonic diagnostic apparatus according to claim 1, wherein a groove portion for holding wiring is provided in the second connector.

9. An ultrasonic diagnostic apparatus comprising:
an ultrasonic transducer including an acoustic lens, an electromechanical conversion element, a backing material, and a housing;
a transducer holder that is driven to rotate in a state of holding the ultrasonic transducer;
a cap arranged on an outer side of the ultrasonic transducer;
an acoustic medium filled in the cap;
a first connector fixed in an inside of the ultrasonic transducer; and
a second connector that is arranged in the transducer holder, movable in a direction perpendicular to a connecting direction in the transducer holder in a state before connector connection, and connectable to the first connector, wherein
the transducer holder has an external shape corresponding to an external shape of the ultrasonic transducer,
the ultrasonic transducer is elastically deformed on the transducer holder side thereof with respect to the transducer holder and fit in the transducer holder to be positioned and fixed, and
the second connector is positioned by connection to the first connector to electrically connect the ultrasonic transducer.

10. An ultrasonic diagnostic apparatus comprising:
an ultrasonic transducer including an acoustic lens, an electromechanical conversion element, a backing material, and a housing having a cylindrical external shape portion;
a transducer holder that has a cylindrical inner peripheral surface shape portion corresponding to the cylindrical external shape of the housing and holds the ultrasonic transducer;
a first connector fixed in an inside of the ultrasonic transducer; and
a second connector that is arranged in the transducer holder and supported in a state movable in a direction perpendicular to a connector connecting direction in the transducer holder in a state before connector connection, wherein
in the ultrasonic transducer or the transducer holder, a positioning convex portion or concave portion for determining a position in an ultrasound generating direction of the ultrasonic transducer is formed,
in the ultrasonic transducer and the transducer holder, the transducer holder side is elastically deformed, the cylindrical external shape portion of the ultrasonic transducer fits in the cylindrical inner peripheral surface shape portion of the transducer holder via the positioning convex portion or concave portion, positioning and fixing in the ultrasound generating direction of the ultrasonic transducer are performed, and
the first connector and the second connector are connected to electrically connect the ultrasonic transducer.

11. An ultrasonic diagnostic apparatus comprising:
an ultrasonic transducer including an acoustic lens, an electromechanical conversion element, a backing material, and a housing having a cylindrical external shape portion;
a transducer holder that has a cylindrical inner peripheral surface shape portion corresponding to a cylindrical external shape of the housing and holds the ultrasonic transducer;
a first connector fixed in an inside of the ultrasonic transducer; and
a second connector that is arranged in the transducer holder and movable in a direction perpendicular to a connector connecting direction in the transducer holder in a state before connector connection, wherein
in the ultrasonic transducer, a convex step portion for determining a position in an ultrasound generating direction of the ultrasonic transducer is provided,
the transducer holder has a fitting portion corresponding to the step portion,
in fitting of the ultrasonic transducer and the transducer holder, the transducer holder is elastically deformed and the cylindrical external shape portion of the ultrasonic transducer fits in the cylindrical inner peripheral surface shape portion of the transducer holder in the step portion to be positioned and fixed, and
the first connector and the second connector are connected to electrically connect the ultrasonic transducer.

12. An ultrasonic diagnostic apparatus comprising:
an ultrasonic transducer including an acoustic lens, an electromechanical conversion element, a backing material, and a housing;
a first connector fixed in an inside of the ultrasonic transducer; and
a second connector connectable to the first connector, wherein
a ground terminal of the first connector is electrically and mechanically fixed to the housing on an outer side of the ultrasonic transducer.

13. An ultrasonic diagnostic apparatus comprising:
an ultrasonic transducer including an acoustic lens, an electromechanical conversion element, a backing material, and a housing;
a first connector fixed in an inside of the ultrasonic transducer; and
a second connector connectable to the first connector, wherein
a wiring portion of the first connector to the ultrasonic transducer is covered with a protection member in order to prevent contact of an electric connection portion with an adjacent terminal.

14. An ultrasonic diagnostic apparatus comprising:
an ultrasonic transducer including an acoustic lens, an electromechanical conversion element, a backing material, and a housing;
a first connector fixed in an inside of the ultrasonic transducer; and
a second connector connectable to the first connector, wherein
a positioning portion for performing positioning of the ultrasonic transducer with respect to the housing is provided on an external shape portion of the first connector.
